(19) United States
(12) Patent Application Publication
(10) Pub. No.: US 2023/0237817 A1
(43) Pub. Date: Jul. 27, 2023

(54) CULTURING ASSISTANCE DEVICE, CULTURING ASSISTANCE METHOD, AND NON-TRANSITORY COMPUTER-READABLE MEDIUM

(71) Applicant: NIKON CORPORATION, Tokyo (JP)

(72) Inventors: Naoya OTANI, Yokohama-shi (JP); Yuuya TAKAYAMA, Foster City, CA (US); Tetsuya KOIKE, Yamoto-shi (JP)

(73) Assignee: NIKON CORPORATION, Tokyo (JP)

(21) Appl. No.: 18/151,765

(22) Filed: Jan. 9, 2023

Related U.S. Application Data
(63) Continuation of application No. PCT/JP2021/025104, filed on Jul. 2, 2021.
(60) Provisional application No. 63/050,885, filed on Jul. 13, 2020.

(51) Int. Cl. G06V 20/69

(52) U.S. Cl. CPC G06V 20/695

(57) ABSTRACT

There is provided a culturing assistance device including: a memory which stores a program; and a processor which executes the program, wherein the processor executes the program to implement: acquiring a plurality of captured images in which cells are captured in time series; calculating a probability that a state of the cells shown in the acquired image is a certain state; reading predetermined state change rule information which is stored in a storage unit and indicates a relationship among a plurality of states of the cells in the time-series; and determining the state of the cell based on the calculated probability and the read state change rule information.

Priority is claimed on U.S. Patent Application No. 63/050,885, provisionally filed Jul. 13, 2020, the content of which is incorporated herein by reference.

### TECHNICAL FIELD

The present invention relates to a culturing assistance device, a culturing assistance method, and a non-transitory computer-readable medium.

### BACKGROUND ART

A technology is known for assisting cell culture by determining the state of the degree of cell growth from an image in which cells are captured. As an example, there is known a cell culture state evaluation device that identifies cells by tracking images in which cells are captured in time series, and obtains parameters representing the culture state of cells (Patent Document 1). It is required to be able to determine the state of cell growth with higher accuracy.

### CITATION LIST

### Patent Document

### [Patent Document 1]

Japanese Unexamined Patent Application, First Publication No. 2019-4795

### SUMMARY OF INVENTION

### Technical Problem

According to as aspect of the present invention, there is provided a culturing assistance device including: a memory which stores a program; and a processor which executes the program, wherein the processor executes the program to implement: acquiring a plurality of captured images in which cells are captured in time series; calculating a probability that a state of the cells shown in the acquired image is a certain state; reading predetermined state change rule information which is stored in a storage unit and indicates a relationship among a plurality of states of the cells in the time-series; and determining the state of the cell based on the calculated probability and the read state change rule information.

According to another aspect of the present invention, there is provided a culturing assistance method including: acquiring a plurality of captured images in which cells are captured in time series; calculating a probability that a state of the cells shown in the captured image is a certain state; reading predetermined state change rule information which is stored in a storage unit and indicates a relationship among a plurality of states of the cells in the time-series; and determining the state of the cell based on the calculated probability and the read state change rule information.

According to still another aspect of the present invention, there is provided a non-transitory computer-readable medium storing a program causing a computer to implement: acquiring a plurality of captured images in which cells are captured in time series; calculating a probability that a state of the cells shown in the captured image acquired is a certain state; reading predetermined state change rule information which is stored in a storage unit and indicates a relationship among a plurality of states of the cells in the time series; and determining the state based on the calculated probability and the read state change rule information.

### BRIEF DESCRIPTION OF DRAWINGS

FIG. 1 is a diagram showing an example of a captured image according to an embodiment of the present invention.

FIG. 2 is a diagram showing an example of a captured image according to the embodiment of the present invention.

FIG. 3 is a diagram showing an example of a configuration of a culturing assistance device according to the embodiment of the present invention.

FIG. 4 is a diagram showing an example of a region and a state probability according to the embodiment of the present invention.

FIG. 5 is a diagram showing an example of a state probability time series according to the embodiment of the present invention.

FIG. 6 is a diagram showing an example of state change rule information according to the embodiment of the present invention.

FIG. 7 is a diagram showing an example of state determination processing according to the embodiment of the present invention.

FIG. 8 is a diagram showing an example of regions for each imaging time according to the embodiment of the present invention.

FIG. 9 is a diagram showing an example of maximum likelihood change history calculation processing according to the embodiment of the present invention.

### DESCRIPTION OF EMBODIMENTS

### Embodiment

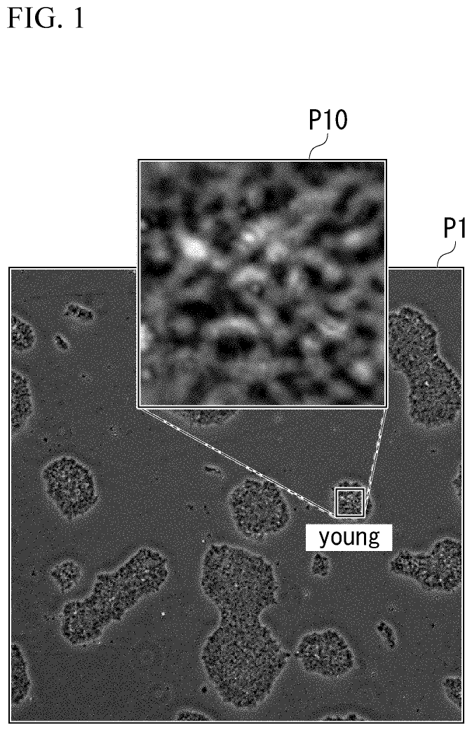

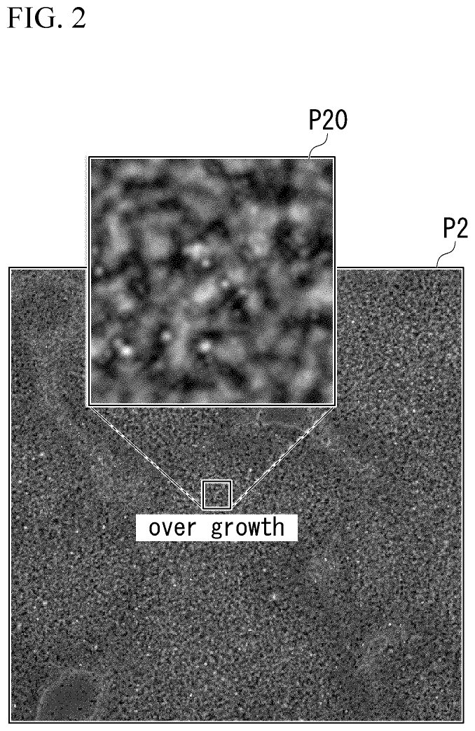

Embodiments of the present invention will be described in detail below with reference to the drawings. FIGS. 1 and 2 are diagrams showing an example of a captured image P according to the present embodiment. In the captured image P, cells in culture are captured. In a captured image P1 shown in FIG. 1, cells of which the degree of cell growth is young are captured. On the other hand, in a captured image P2 shown in FIG. 2, cells of which the degree of cell growth is overgrowth are captured.

In the captured image P1, colonies of young cells are formed in a region R1. An enlarged image P10 is an image obtained by enlarging the region R1. In the captured image P2, colonies of overgrowth cells are formed in a region R2. An enlarged image P20 is an image obtained by enlarging the region R2.

As shown in the drawing, the texture of the enlarged image P10 and the texture of the enlarged image P20 are similar. That is, in the enlarged image P10 and the enlarged image P20, the texture of the young cell image and the texture of the overgrowth cell image are similar.

Similar to the enlarged image P10 and the enlarged image P20, even when the textures are similar, the degree of cell growth of the captured cells may differ. When images with different degrees of cell growth have similar textures, it is difficult to accurately determine the degree of cell growth based on the texture of the still image.

In the related art, there is a method of analyzing the texture of a still image using a machine learning technology such as deep learning to determine the degree of cell growth for each region of the still image. However, when a still image is used, the time-series information of the state change is not used for determination, and the accuracy of determination is not sufficient.

### (Configuration of Culturing Assistance Device)

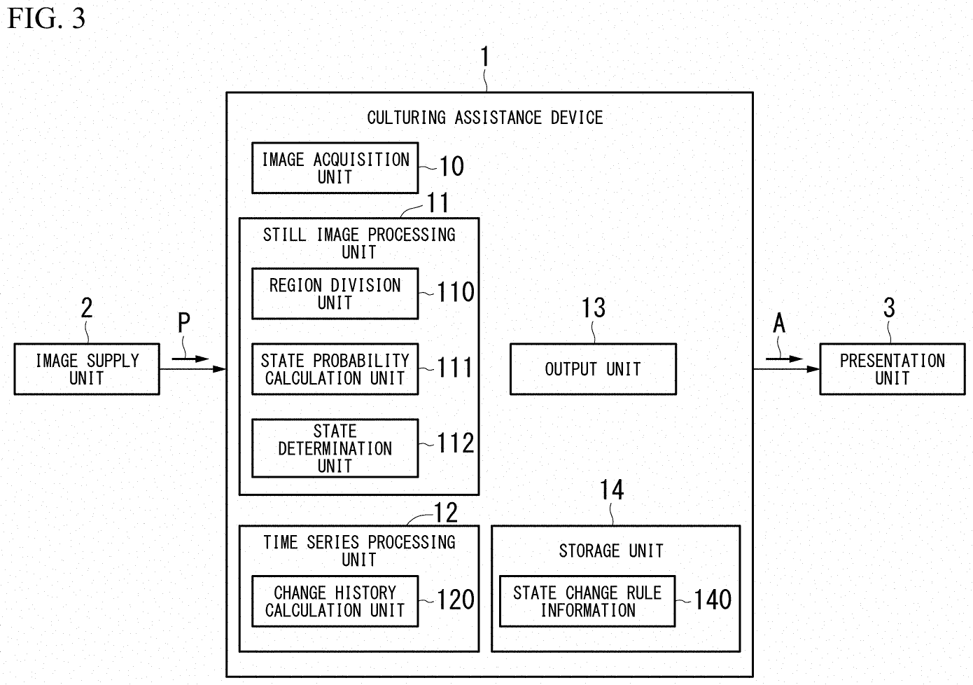

FIG. 3 is a diagram showing an example of a configuration of a culturing assistance device 1 according to the present embodiment. The culturing assistance device 1 uses a plurality of captured images in which cells are captured in time series to determine the cell state based on the change history which is the time-series change of the state of cells.

The culturing assistance device 1 determines a state x regarding the degree of cell growth as the state of cells. The state x is classified into, for example, three states of “young,” “ripe,” and “overgrowth.” That is, the state x includes a young state, a ripe state, and an overgrowth state.

The culturing assistance device 1 includes an image acquisition unit 10, a still image processing unit 11, a time series processing unit 12, an output unit 13, and a storage unit 14. The culturing assistance device 1 is, for example, a computer. The image acquisition unit 10, the still image processing unit 11, the time series processing unit 12, and the output unit 13 are modules realized by executing processing by a central processing unit (CPU) reading a program from a read only memory (ROM).

The image acquisition unit 10 acquires a plurality of captured images P supplied from the image supply unit 2. The plurality of captured images P are time-lapse images in which cells are captured in time series from the start of culturing. That is, the plurality of captured images P are a plurality of images in which cells are captured in time series. Each of the plurality of captured images P is a still image.

Here, the plurality of captured images P are generated by, for example, time-lapse imaging of cells being cultured every 24 hours. The captured image P and the imaging time t, which is the time when the cell was imaged, correspond to each other.

In the captured image P, cells are observed and captured at a predetermined magnification with a time-lapse microscope. Here, the predetermined magnification is set according to the cell type. It is preferable that the predetermined magnification be a magnification such that the size of one cell colony is at least approximately 50 pixels. The predetermined magnification is, for example, a low magnification of approximately 20 times to 40 times. In the culturing assistance device 1, even at a low magnification of approximately 20 times to 40 times, the state of cells is determined with higher accuracy than when a still image is analyzed.

The still image processing unit 11 performs various types of processing on each of the plurality of captured images P. The still image processing unit 11 includes a region division unit 110, a state probability calculation unit 111, and a state determination unit 112.

The region division unit 110 divides the captured image P into a plurality of regions R.

The state probability calculation unit 111 calculates a state probability PR for each of the plurality of captured images P for each of the plurality of regions R of the captured image P divided by the region division unit 110. Here, the state probability PR is a probability that the state of cells shown in the captured image P is the state x. The state probability calculation unit 111 calculates the state probability PR for each of the plurality of states x. In the following, the probability that the state of cells is a certain state x is indicated by state probabilities PRx, i, j, and the like, in the i-th region Ri of a captured image P-j captured at an imaging time tj.

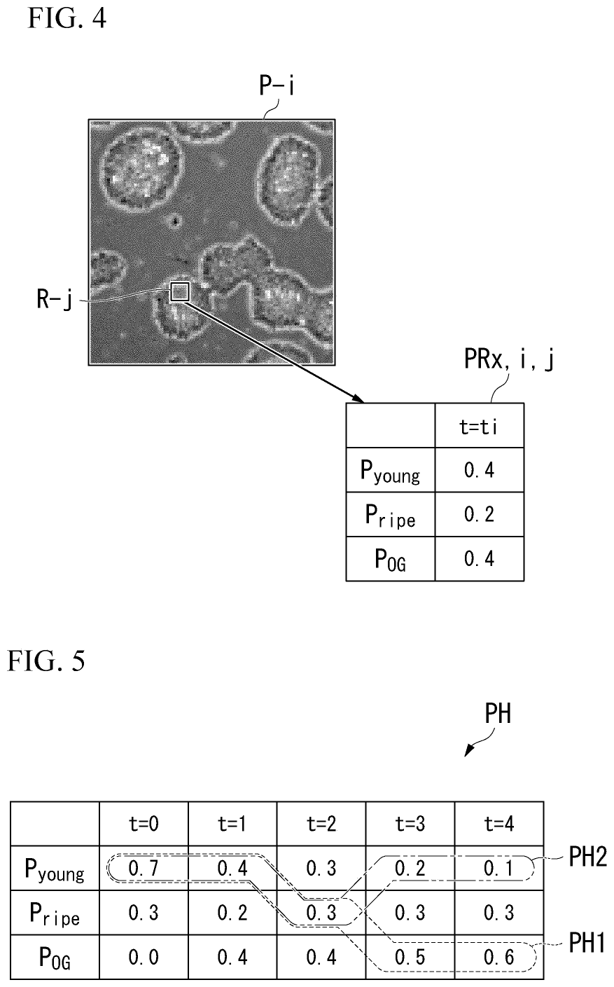

Now referring to FIG. 4, the region R and the state probability PR will be described. FIG. 4 is a diagram showing an example of the region R and the state probability PR according to the present embodiment. FIG. 4 shows, as an example, a region R-j among the plurality of regions R into which the captured image P-i captured at the imaging time ti in the captured image P is divided.

The state probabilities PRx, i, j shown in FIG. 4 are examples of the state probability PR, and the state probabilities PRx, i, j indicate the probabilities that the state of cells is the certain state x at the imaging time ti for the region R-j.

As described above, the plurality of states are “young,” “ripe,” and “overgrowth.” With the state probabilities PRx, i, j shown in FIG. 4, the probability that the state of cells is “young” is 0.4, the probability that the state of cells is “ripe” is 0.2, and the probability that the state of cells is “overgrowth” is 0.4.

Returning to FIG. 3, the description of the configuration of the culturing assistance device 1 is continued.

The state determination unit 112 determines the state x based on a change history H calculated by a change history calculation unit 120 of the time series processing unit 12. The state determination unit 112 determines whether the state x is “young,” “ripe,” or “overgrowth.”

The time series processing unit 12 performs various types of processing on the time series of the plurality of captured images P. The time series processing unit 12 includes the change history calculation unit 120.

The change history calculation unit 120 arranges the state probabilities PR calculated by the state probability calculation unit 111 in time series to calculate a state probability time series PH. Here, the state probability time series PH is a time-series change of the state probability PR. Since the state probability PR is the probability that the state of cells is a certain state x, the state probability time series PH corresponds to the probability that the time-series change of the state x occurs. Hereinafter, the time-series change of the state x will be referred to as the change history H. As described above, the state probability time series PH and the change history H correspond to each other.

The change history calculation unit 120 determines a maximum likelihood change history HL, which is the maximum likelihood time-series change of the state x, based on the state change rule information 140 and the calculated state probability time series PH. The state change rule information 140 indicates a predetermined rule that indicates the relationship between the plurality of states x.

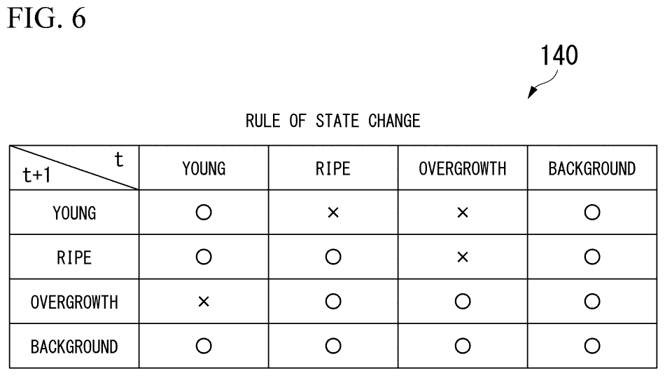

Here, the state probability time series PH and the state change rule information 140 will be described with reference to FIGS. 5 and 6.

FIG. 5 is a diagram showing an example of the state probability time series PH according to the present embodiment. The state probability time series PH shown in FIG. 5 is a time-series change in the state probability PR for five imaging times from imaging time t=0 to imaging time t=4.

A state probability time series PH1 and a state probability time series PH2 are examples of the state probability time series PH. The state probability time series PH1 is a probability for each imaging time for a change history H1 in which the state x changes from imaging time t=0 to imaging time t=4 as “young,” “young,” “ripe,” “overgrowth,” and “overgrowth.” The state probability time series PH2 is the probability for each imaging time for a change history H2 in which the state x changes from imaging time t=0 to imaging time t=4 as “young,” “young,” “ripe,” “young,” and “young.”

The number of state probability time series PH, that is, the number of change histories H, is equal to the number of types of the state x raised to the power of the number of imaging times T. The state probability time series PH shown in FIG. 5 includes the state probability time series PH1 and the state probability time series PH2, and only 243 state probability time series are included, which is the number of types of the state x, that is, 3 to the power of 5.

FIG. 6 is a diagram showing an example of the state change rule information 140 according to the present embodiment.

The state change rule information 140 indicates the relationship between the state x at the first time included in the time series of the imaging time T and the state x at the second time included in the time series of the imaging time T. Here, the state x includes a first state and a second state different from the first state, and the state change rule information 140 indicates that the state x at the second time is not the second state when the state x at the first time is the first state.

In the present embodiment, the first time is a certain imaging time t, and the second time is an imaging time t+1 after the imaging time t. The imaging time t+1 is the time 24 hours after the imaging time t.

In addition, in the state change rule information 140, the state x includes “background” which is a state when the region R corresponds to a background other than cells.

For example, the state change rule information 140 indicates that the state x at the imaging time t+1 is not “overgrowth” when the state x at the imaging time t is “young.” The state change rule information 140 indicates that the state x at the imaging time t+1 may be “young,” “ripe,” or “background” when the state x at the imaging time t is “young.”

Further, the state change rule information 140 indicates that the state x at the imaging time t+1 is not “young” when the state x at the imaging time t is “ripe.” The state change rule information 140 indicates that the state x at the imaging time t+1 may be “ripe,” “overgrowth,” or “background” when the state x at the imaging time t is “ripe.”

Further, the state change rule information 140 indicates that the state x at the imaging time t+1 is not “young” or “ripe” when the state x at the imaging time t is “overgrowth.” The state change rule information 140 indicates that the state x at the imaging time t+1 may be “overgrowth” or “background” when the state x at the imaging time t is “overgrowth.”

Returning to FIG. 3, the description of the configuration of the culturing assistance device 1 is continued.

The output unit 13 outputs to the presentation unit 3 a determination result A, which is the result of determination of the state x by the state determination unit 112.

The storage unit 14 stores the state change rule information 140.

The image supply unit 2 captures the captured image P and supplies the captured image P to the culturing assistance device 1. The image supply unit 2 is, for example, a time-lapse microscope.

The presentation unit 3 presents the determination result A output by the culturing assistance device 1. The presentation unit 3 is, for example, a display.

### (Processing of Culturing Assistance Device)

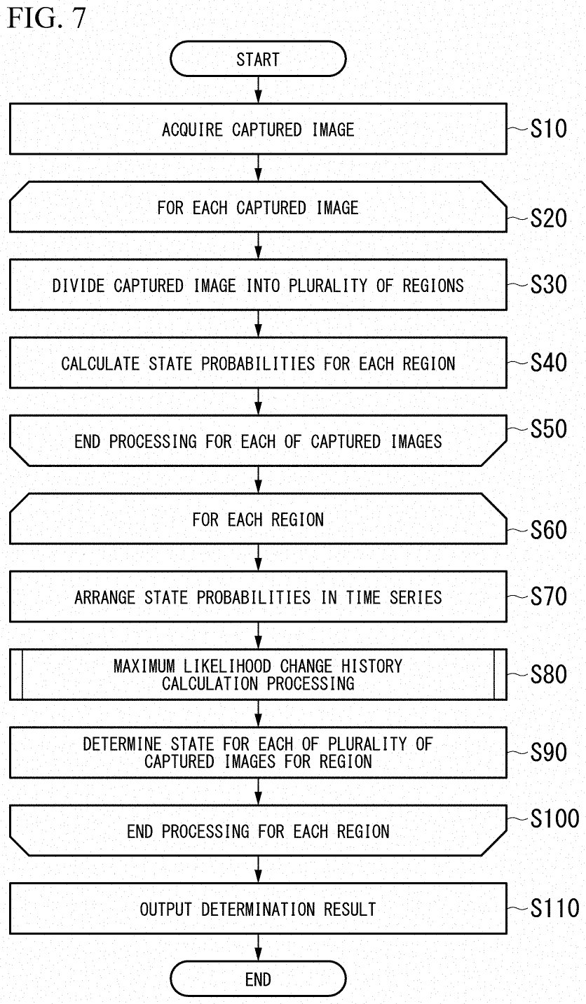

With reference to FIG. 7, the state determination processing, which is the processing in which the culturing assistance device 1 determines the state of cells shown in the captured image P, will be described. FIG. 7 is a diagram showing an example of the state determination processing according to the present embodiment.

Step S10: The image acquisition unit 10 acquires a plurality of captured images P supplied from the image supply unit 2. The image acquisition unit 10 supplies the plurality of acquired captured images P to the still image processing unit 11.

In addition, during the culturing process, the position of the entire colony of cells may shift due to detachment of the cells from the well, and the position of the entire colony of cells may differ between a plurality of captured images P. When the positions of the entire colony of cells are different between the plurality of captured images P, the still image processing unit 11 may perform, by image processing, alignment processing such that the positions of the entire colony of cells are common among the plurality of captured images P.

Step S20: The still image processing unit 11 starts processing repeated for each of the captured images P.

Step S30: The region division unit 110 divides the captured image P supplied from the image acquisition unit 10 into a plurality of regions R. As an example, the region division unit 110 divides the captured image P into a plurality of regions R in units of one pixel.

Note that the region division unit 110 may divide the captured image P into a plurality of regions R with each region formed of a plurality of pixels as a unit. That is, the region R is a region based on the pixels of the captured image P. A region composed of a plurality of pixels is, for example, a square or a rectangle.

Step S40: The state probability calculation unit 111 calculates the state probability PR for each of the plurality of regions R of the captured images P divided by the region division unit 110. Here, the state probability calculation unit 111 calculates the state probability PR for each state x. The state probability calculation unit 111 supplies the calculated state probability PR to the time series processing unit 12.

As an example, the state probability calculation unit 111 calculates the state probability PR based on machine learning. The state probability calculation unit 111 calculates the state probability PR for each pixel of the captured image P using a convolutional neural network (CNN) as an example of machine learning.

As described above, the state probability calculation unit 111 calculates the state probability PR that the state x of cells shown in the captured image P acquired by the image acquisition unit 10 is a certain state for each of the plurality of states and for each of the plurality of captured images P. Here, the state probability calculation unit 111 calculates the state probability PR that the state x of cells shown in the captured image P acquired by the image acquisition unit 10 is a certain state for each of the plurality of states and for each of the plurality of captured images P, for a predetermined region of the captured image P acquired by the image acquisition unit 10.

Step S50: The still image processing unit 11 ends processing repeated for each of the captured images P.

Step S60: The time series processing unit 12 starts processing repeated for each region R.

Step S70: The change history calculation unit 120 arranges the state probabilities PR calculated by the state probability calculation unit 111 in time series for each state x. Here, among the state probabilities PR calculated by the state probability calculation unit 111, the change history calculation unit 120 arranges the state probabilities PRx, i, j for the region Ri in time series to form a set. j is a label corresponding to the imaging time T. The region Ri is a region at a common position for each of the plurality of captured images P.

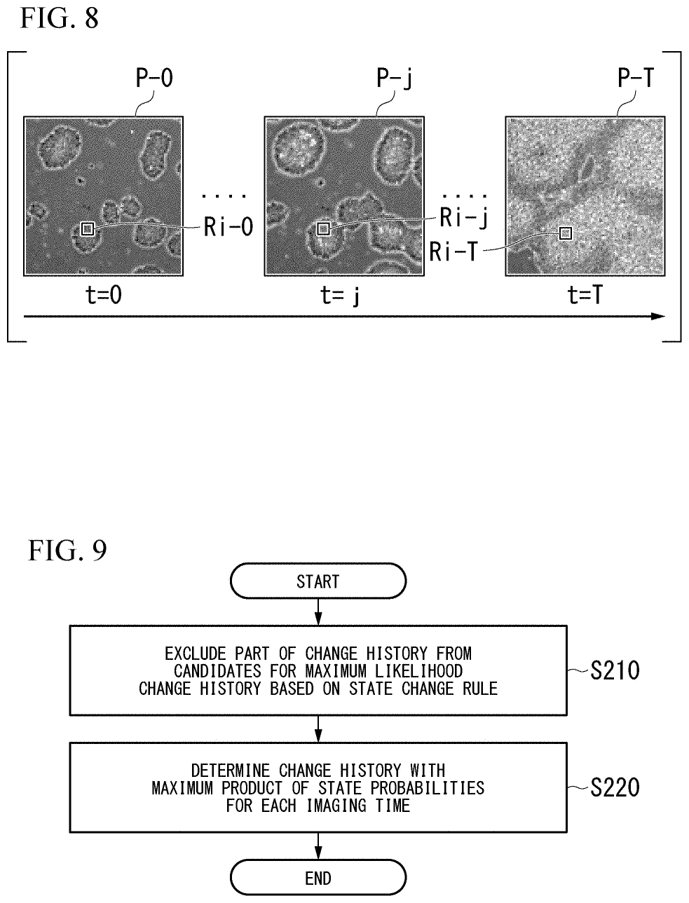

Here, with reference to FIG. 8, the region Ri for each imaging time T will be described. FIG. 8 is a diagram showing an example of the regions Ri for each imaging time T according to the present embodiment. In FIG. 8, among the plurality of captured images P, a captured image P-0 captured at the start of culture, a captured image P-j captured after a predetermined time has passed since the start of culture, and a captured image P-T captured at the end of culture are shown.

The region Ri-0, the region Ri-j, and the region Ri-T are examples of regions Ri of each of the captured image P-0, the captured image P-j, and the captured image P-T. The region Ri-0, the region Ri-j, and the region Ri-T are common places of each of the captured image P-0, the captured image P-j, and the captured image P-T.

Returning to FIG. 7, the description of the state determination processing is continued.

The change history calculation unit 120 calculates the result of arranging the state probabilities PR in time series as the state probability time series PH. The state probability time series PH is a set in which the state probabilities PRx, i, j of the number of imaging times T, that is, the number of captured images P, are arranged in the order of the label j indicating the imaging time T to form a set.

Step S80: The change history calculation unit 120 executes maximum likelihood change history calculation processing, which is processing for calculating the maximum likelihood change history HL.

Here, with reference to FIG. 9, details of the maximum likelihood change history calculation processing will be described. FIG. 9 is a diagram showing an example of the maximum likelihood change history calculation processing according to the present embodiment. The processing of steps S210 and S220 in FIG. 9 corresponds to the processing of step S80 in FIG. 7.

Step S210: Based on the state change rule information 140, the change history calculation unit 120 excludes a part of the change history H from candidates for the maximum likelihood change history HL. Here, the change history H is the change history H corresponding to the state probability time series PH calculated by the change history calculation unit 120 in step S70. The change history calculation unit 120 reads the state change rule information 140 from the storage unit 14.

Based on the state change rule information 140 shown in FIG. 6, the change history calculation unit 120 excludes, for example, the change history H in which the state x at the imaging time t+1 is “overgrowth” when the state x at the imaging time t is “young,” the change history H in which the state x at the imaging time t+1 is “young” when the state x at the imaging time t is “ripe”, and the change history H in which the state x is “young” or “ripe” at the imaging time t+1 when the state x at the imaging time t is “overgrowth”, from candidates for the maximum likelihood change history HL.

Step S220: The change history calculation unit 120 determines the change history H with the maximum product of the state probabilities PR for each imaging time T. The change history calculation unit 120 calculates, for the change history H, the product of the state probabilities PR for each imaging time T indicated by the state probability time series PH corresponding to this change history H. The change history calculation unit 120 compares the calculated products among the change histories H, and determines the change history H with the maximum calculated product.

The change history calculation unit 120 determines the change history H with the maximum determined product as the maximum likelihood change history HL. The change history calculation unit 120 supplies the determined maximum likelihood change history HL to the state determination unit 112.

When determining the correct state probability PR, when the determination cannot be made using the state probability PR at a certain imaging time as a reference, the change history calculation unit 120 may make a determination with reference to the state probability PR at the imaging time after the imaging time. For example, in FIG. 5, two cases of 30% of state probabilities appear at imaging time t=2. Therefore, when determining the maximum likelihood change history HL, the change history calculation unit 120 cannot determine at the point of time t=2. Even in that case, when the change history calculation unit 120 refers to the state probability PR of 50% for the “overgrowth POG” at the point of time that is the imaging time t=3, the state probability PR is 30% at the point of time that is the imaging time t=2 can be determined to be “ripe Pripe.”

As described above, the change history calculation unit 120 calculates the maximum likelihood change history HL, which is the maximum likelihood time-series change of the state x in the change history H. Here, the maximum likelihood time-series change is a time-series change that maximizes the product of the state probabilities PR for each imaging time indicated by the time series of the state probabilities. Further, the change history calculation unit 120 excludes the change histories H that are not candidates for the maximum likelihood change history HL from the plurality of change histories H based on the state change rule information 140, and then calculates the maximum likelihood change history HL based on the state probability PR calculated by the state probability calculation unit 111. In addition, since the change history H increases exponentially with respect to the number of imaging times, it is inefficient to calculate and compare the product of the state probabilities PR from each change history H when the number of imaging times is large. Therefore, a dynamic programming algorithm such as the Viterbi algorithm may be used as a method for efficiently obtaining the maximum likelihood change history HL.

Returning to FIG. 7, the description of the state determination processing is continued.

Step S90: Based on the maximum likelihood change history HL determined by the change history calculation unit 120, the state determination unit 112 determines the state x for each of the plurality of captured images P for the region Ri. The state determination unit 112 extracts the state corresponding to the imaging time t, which is the imaging time of the captured image P-t, from the maximum likelihood change history HL. The state determination unit 112 determines the state extracted from the maximum likelihood change history HL as the state x. For example, when the state extracted from the maximum likelihood change history HL is “young,” the state determination unit 112 determines “young” as the state x.

Step S100: The state determination unit 112 ends the processing repeated for each region R.

Specifically, the state determination unit 112 converts the determination result A determined as the state x into the change history H1 (“young,” “young,” “ripe,” “overgrowth,” and “overgrowth”) of the state x corresponding to the maximum likelihood change history HL and output it to the output unit 13.

Step S110: The output unit 13 outputs the determination result A to the presentation unit 3. The presentation unit 3 displays and presents the determination result A on the display. In addition, the output unit 13 may store the determination result A in the storage unit 14 and output the determination result A to the external device when requested by the external device.

When the determination result A is displayed on the display (an example of the display unit), the presentation unit 3 may display “young,” “ripe,” and “overgrowth,” as the state x of the region Ri only for the captured image P at a certain imaging time t, based on the operation of the presentation unit 3 by the user. Further, the presentation unit 3 may sequentially display the determination result A for the captured images P captured at different imaging times based on the operation of the user on the presentation unit 3. Further, the presentation unit 3 may display “young,” “ripe,” and “overgrowth” in time series for the desired region Ri of the captured image P for each imaging time t. The desired region Ri is designated by the operation of the user on the presentation unit 3.

Further, when displaying the determination result A on the display, the presentation unit 3 may indicate all the state probabilities PR for each imaging time t as shown in FIG. 5, and may highlight and present the state probability time series PH1 corresponding to the maximum likelihood change history HL, which indicates the determination result A.

In addition, when a captured image captured at a new imaging time is added and the state determination processing described above is executed, a partial change history obtained by excluding the change history corresponding to the new imaging time from the calculated maximum likelihood change history HL, and the maximum likelihood change history HL calculated before the captured image captured at the new imaging time is added may be different from each other.

Specifically, in the change history H-N corresponding to the imaging time a to the imaging time tN, even the change history H1-N in which the product of the state probability PR for each imaging time T indicated by the state probability time series PH is the maximum may be excluded based on the state change rule information 140 by adding the state x at the imaging time tN+1. By excluding the change history H1-N with the maximum product of the state probabilities PR from the imaging time t1 to the imaging time tN, the change history H1-2, which is the second largest after the change history H1-N, from which the product of the state probabilities PR is excluded from the imaging time t1 to the imaging time tN, may be included in the maximum likelihood change history HL-N+1 from the imaging time t1 to the imaging time tN+1 as a part corresponding to the imaging time t1 to the imaging time tN.

Therefore, when determining the state x of cells for the captured image P-j captured at a certain imaging time tj, each time the captured image P captured after the imaging time tj is added to the state determination processing, the determination result of the state x of cells for the image P-j may be different. When determining the state x of cells for the captured image P-j captured at a certain imaging time tj, it is considered that the accuracy of determining the state x of cells for the captured image Pj is increased when more captured images P captured after the imaging time tj as the culture progresses are added to the state determination processing.

In step S90 described above, an example of the case where the state determination unit 112 determines the state x for each of the plurality of captured images P has been described, but the present invention is not limited to this. The state determination unit 112 may determine the state x for one captured image P-i captured at a certain imaging time among the plurality of captured images P.

In the present embodiment, an example in which the change history calculation unit 120 calculates the maximum likelihood change history HL, which is the maximum likelihood time-series change of the state x, has been described, but the present invention is not limited to this. Instead of the maximum likelihood change history HL, the change history calculation unit 120 may calculate the change history H having the second largest product of the state probabilities PR for each imaging time T indicated by the state probability time series PH.

Further, instead of the maximum likelihood change history HL, the change history calculation unit 120 may calculate the change history H having the smallest product of the state probabilities PR for each imaging time T indicated by the state probability time series PH. The change history H with the smallest product of the state probabilities PR for each imaging time T may be used, for example, to preliminarily exclude the state with the lowest probability when the determination processing for determining the state x is performed in an external device. This determination processing is, for example, processing based on machine learning.

As described above, the change history calculation unit 120 calculates the change history H, which is the time-series change of the state x, based on the state change rule information 140 and the state probability PR calculated by the state probability calculation unit 111. Here, the change history calculation unit 120 calculates the change history H for the region R.

In the above-described embodiment, an example in which the maximum likelihood change history calculation processing is executed in step S80 has been described, but the present invention is not limited to this. In step S80, the change history calculation unit 120 may calculate the maximum likelihood change history HL based on dynamic programming instead of the maximum likelihood change history calculation processing.

In the above-described embodiment, an example of the case where, in step S210, the change history calculation unit 120 excludes a part of the change history H from the candidates for the maximum likelihood change history HL based on the state change rule information 140 has been described, but the present invention is not limited to this. The change history calculation unit 120 may change the value of the state probability PR included in the state probability time series PH corresponding to the change history H based on the state change rule information 140.

For example, based on the state change rule information 140, the change history calculation unit 120 reduces the state probability PR that the state x is “overgrowth” at the imaging time t+1 of the state probability time series PH corresponding to this change history H by a predetermined ratio, for the change history H in which the state x at the imaging time t+1 is “overgrowth” when the state x at the imaging time t is “young.” Here, the state change rule information 140 increases the “young” state probability PR and the “ripe” state probability PR such that the sum of the state probabilities PR of each state x at the imaging time t+1 is preserved.

In addition, in the above-described embodiment, an example of the case where the determination is made when the captured image P is divided into the plurality of regions R has been described, but the present invention is not limited to this. The entire captured image P may be selected as the region R. When the entire captured image P is selected as the region R, the culturing assistance device 1 determines the state x for the captured image P.

In the above-described embodiment, an example of the case where the state x is one of three types of cell growth, that is, “young,” “ripe,” and “overgrowth,” has been described, but the present invention is not limited to this. The state x may be of more than three types, including a state intermediate between “young” and “ripe” and a state intermediate between “ripe” and “overgrowth.” In addition, the state x may be classified by a numerical value indicating the degree of cell growth.

Further, the state x may be an index of the state of cells other than the degree of growth. The state x may be, for example, each state in a process in which a normal cell changes into a cancer cell.

### SUMMARY

As described above, the culturing assistance device 1 according to the present embodiment includes the image acquisition unit 10, the state probability calculation unit 111, the change history calculation unit 120, and the state determination unit 112.

The image acquisition unit 10 acquires the plurality of captured images P in which cells are captured in time series.

The state probability calculation unit 111 calculates the probability that the state of cells shown in the captured image P acquired by the image acquisition unit 10 is a certain state (the state probability PR in this example) for each of the plurality of states x (“young,” “ripe,” and “overgrowth” which are the degrees of cell growth in this example) and for each of the plurality of captured images P.

The change history calculation unit 120 calculates the change history H, which is the time-series change of the state x, based on a predetermined rule (the state change rule information 140 in this example) indicating the relationship between the plurality of states x and a probability calculated by the state probability calculation unit 111 (the state probability PR in this example).

The state determination unit 112 determines the state x based on the change history H calculated by the change history calculation unit 120.

With this configuration, the culturing assistance device 1 according to the present embodiment, when determining the state of cells using the captured image of the cells, can determine the state of cells based on the change history, which is the time-series change of the state of cells, and thus the state can be determined more accurately than when a still image is used.

In addition, in the culturing assistance device 1 according to the present embodiment, the change history calculation unit 120 calculates the maximum likelihood change history HL, which is the maximum likelihood time-series change of the state x in the change history H.

With this configuration, the culturing assistance device 1 according to the present embodiment can determine the state of cells based on the maximum likelihood change history HL, which is the maximum likelihood time-series change of the state x in the change history H, and thus the state of cells can be determined more accurately than when not based on the history HL.

In addition, in the culturing assistance device 1 according to the present embodiment, the change history calculation unit 120 calculates the maximum likelihood change history HL based on the probability calculated by the state probability calculation unit 111 (the state probability PR in this example) after excluding the change histories H that are not candidates for the maximum likelihood change history HL from the plurality of change histories H, based on a predetermined rule (the state change rule information 140 in this example) indicating the relationship between the plurality of states x.

With this configuration, the culturing assistance device 1 according to the present embodiment can calculate the maximum likelihood change history HL by excluding the change histories H which are not candidates for the maximum likelihood change history HL from the plurality of change histories H based on a predetermined rule (the state change rule information 140 in this example) indicating the relationship between the plurality of states x, and thus can determine the state of cells more accurately than when the maximum likelihood change history HL is calculated based on the probability (the state probability PR in this example).

Further, in the culturing assistance device 1 according to the present embodiment, a predetermined rule (the state change rule information 140 in this example) indicating the relationship between the plurality of states x indicates the relationship between the state x at the first time included in the time series and the state x at the second time included in the time series.

With this configuration, the culturing assistance device 1 according to the present embodiment can calculate the change history H based on the relationship between the state x at the first time and the state x at the second time, and thus can determine the state of cells more accurately than when not based on the relationship between the state x at the first time and the state x at the second time.

In addition, in the culturing assistance device 1 according to the present embodiment, the state x includes the first state (for example, “young” in this example) and the second state (for example, “overage” in this example) different from the first state, and the predetermined rule (the state change rule information 140 in this example) indicates that the state x at the second time is not the second state (for example, “overgrowth” in this example) when the state x at the first time is the first state (for example, “young” in this example).

With this configuration, in the culturing assistance device 1 according to the present embodiment, when the state x at the first time is the first state (for example, “young” in this example), the change history H can be calculated based on the fact that the state x at the second time is not the second state (for example, “overgrowth” in this example), and thus, when the state x at the first time is the first state (for example, “young” at this example), the state of cells can be determined more accurately than when not based on the fact that the state x at the second time is not the second state (for example, “overgrowth” in this example).

Further, in the culturing assistance device 1 according to the present embodiment, the state x is a state regarding the degree of cell growth.

With this configuration, the culturing assistance device 1 according to the present embodiment, when determining the degree of cell growth using the captured image of the cells, can determine the degree of cell growth based on the change history, which is the time-series change of the degree of the cell growth, and thus the degree of cell growth can be determined more accurately than when a still image is used.

In addition, in the culturing assistance device 1 according to the present embodiment, the state x includes a young state, a ripe state, and an overgrowth state. With this configuration, the culturing assistance device 1 according to the present embodiment can determine which of the state of cells is young, ripe, or overgrowth.

In addition, in the culturing assistance device 1 according to the present embodiment, the state probability calculation unit 111 calculates the probability that the state x of cells shown in the captured image P is a certain state (the state probability PR in this example) for each of the plurality of states x and for each of the plurality of captured images P, for a predetermined region (the region R in this example) of the captured image P acquired by the image acquisition unit 10, the change history calculation unit 120 calculates the change history H for the predetermined region (the region R in this example), and the state determination unit 112 determines the state x for a predetermined region (the region R in this example).

With this configuration, in the culturing assistance device 1 according to the present embodiment, the degree of cell growth can be determined for each predetermined region (the region R in this example), and thus the degree of cell growth can be determined for each predetermined region (the region R in this example) more accurately than when a still image is used.

Further, in the culturing assistance device 1 according to the present embodiment, the predetermined region (the region R in this example) is a region based on the pixels of the captured image P (the region corresponding to one pixel in this example).

With this configuration, in the culturing assistance device 1 according to the present embodiment, the degree of cell growth can be determined in units of regions based on the pixels of the captured image P (regions corresponding to one pixel in this example), and thus the degree of cell growth can be determined for each region (the region corresponding to one pixel in this example) based on the pixels more accurately than when a still image is used.

In addition, in the culturing assistance device 1 according to the present embodiment, the state determination unit 112 determines the state x for each of the plurality of captured images P.

With this configuration, the culturing assistance device 1 according to the present embodiment can determine the state x for each of the plurality of captured images P, and thus it is possible to determine the time-series change of the state x.

In addition, a part of the culturing assistance device 1 in the above-described embodiment, for example, the image acquisition unit 10, the still image processing unit 11, and the time series processing unit 12, may be realized by a computer. In such a case, a program for realizing this control function may be recorded in a computer-readable recording medium, and a program recorded in the recording medium may be read into a computer system and realized by execution. In addition, the “computer system” here is a computer system built into the culturing assistance device 1, and includes hardware such as an OS and peripheral devices. Further, the “computer-readable recording medium” refers to a portable medium such as a flexible disk, a magneto-optical disc, a ROM, and a CD-ROM, and a storage device such as a hard disk installed in a computer system. Further, the “computer-readable recording medium” may include a medium that dynamically retains the program for a short time, such as a communication line when the program is transmitted via a network such as the Internet or a communication line such as a telephone line, and a medium that holds the program for a certain period of time, such as a volatile memory inside a computer system serving as a server or a client in that case. Further, the program may be for realizing a part of the functions described above, or may be capable of realizing the functions described above in combination with a program already recorded in the computer system.

In addition, a part or all of the culturing assistance device 1 in the above-described embodiment may be realized as an integrated circuit such as large scale integration (LSI). Each functional block of the culturing assistance device 1 may be individually processorized, or may be partially or entirely integrated into a processor. Further, the method of circuit integration is not limited to LSI, but may be realized by a dedicated circuit or a general-purpose processor. In addition, when an integration circuit technology that replaces LSI appears due to advances in semiconductor technology, an integrated circuit based on this technology may be used.

Although one embodiment of the present invention has been described in detail above with reference to the drawings, the specific configuration is not limited to the above, and various design changes and the like can be made without departing from the gist of the present invention.

### REFERENCE SIGNS LIST

1 Culturing assistance device
10 Image acquisition unit
111 State probability calculation unit
120 Change history calculation unit
112 State determination unit
P Captured image
H Change history
HL Maximum likelihood change history

What is claimed is:

1. A culturing assistance device comprising:
a memory which stores a program; and
a processor which executes the program, wherein
the processor executes the program to implement:
acquiring a plurality of captured images in which cells are captured in time series;
calculating a probability that a state of the cells shown in the acquired image is a certain state;
reading predetermined state change rule information which is stored in a storage unit and indicates a relationship among a plurality of states of the cells in the time-series; and
determining the state of the cell based on the calculated probability and the read state change rule information.

2. The culturing assistance device according to claim 1, wherein
the processor executes the program to implement: further calculating a change history, which represents a time-series change of the state of the cell, based on the calculated probability and the read state change rule information;
in calculating the probability, the probability for each of the plurality of states of the cells and for each of the plurality of captured images is calculated and
in determining the state of the cell, the state of the cell based on the calculated change history is determined.

3. The culturing assistance device according to claim 2, wherein
the processor executes the program to implement: further displaying the determined state of the cell for each of the plurality of captured images in time series; and
calculates, in calculating the change history, a likelihood based on the probability calculated for each of the captured images, for all candidates of the change history, selects a maximum likelihood change history that is a time-series change of the maximum likelihood of the state of the cell, and outputs the maximum likelihood change history to the presentation unit.

4. The culturing assistance device according to claim 3, wherein,
in calculating the change history, a change history which is not a candidate for the maximum likelihood change history from the plurality of change histories based on the state change rule information is excluded, and then, the maximum likelihood change history is calculated based on the calculated probability.

5. The culturing assistance device according to claim 2, wherein
in calculating the probability, the probability that a state of the cells shown in the captured image is a certain state for each of the plurality of states and for each of the plurality of captured images, for a predetermined region of the acquired captured image is calculated,
in calculating the change history, the change history for the region is calculated, and
in determining the state of the cell, the state of the cell of the region is determined.

6. The culturing assistance device according to claim 5, wherein
the region is a region based on pixels of the captured image.

7. The culturing assistance device according to claim 1, wherein
the state change rule information indicates a relationship between the state of the cell at a first time included in the time series and the state of the cell at a second time included in the time series.

8. The culturing assistance device according to claim 7, wherein
the states include a first state and a second state different from the first state, and
the state change rule information indicates that the state of the cell at the second time is not the second state when the state of the cell at the first time is the first state.

9. The culturing assistance device according to claim 1, wherein
the state of the cell is a state of a degree of growth of the cells.

10. The culturing assistance device according to claim 9, wherein
the state of the cell includes a young state, a ripe state, and an overgrowth state.

11. The culturing assistance device according to claim 1, wherein
in determining the state of the cell, the state of the cell for each of the plurality of captured images is determined.

12. A culturing assistance method comprising:
acquiring a plurality of captured images in which cells are captured in time series;
calculating a probability that a state of the cells shown in the captured image is a certain state;
reading predetermined state change rule information which is stored in a storage unit and indicates a relationship among a plurality of states of the cells in the time-series; and
determining the state of the cell based on the calculated probability and the read state change rule information.

13. The culturing assistance method according to claim 12, further comprising:
calculating a change history, which represents a time-series change of the state of the cell, based on the calculated probability and the read state change rule information;
wherein in calculating the probability, the probability for each of the plurality of states of the cells and for each of the plurality of captured images is calculated and in determining the state of the cell, the state of the cell based on the calculated change history is determined.

14. A non-transitory computer-readable medium storing a program causing a computer to implement:
acquiring a plurality of captured images in which cells are captured in time series;
calculating a probability that a state of the cells shown in the captured image acquired is a certain state;
reading predetermined state change rule information which is stored in a storage unit and indicates a relationship among a plurality of states of the cells in the time series; and
determining the state based on the calculated probability and the read state change rule information.

15. A non-transitory computer-readable medium according to claim 14, wherein
the program causing the computer to implement further: calculating a change history, which represents a time-series change of the state of the cell, based on the calculated probability and the read state change rule information; and
the program causing the computer to implement:
in calculating the probability, calculates the probability for each of the plurality of the states of the cell and for each of the plurality of the captured images; and
in determining the state of the cell, determining the state of the cell based on the calculated change history.
